(19) United States
(12) Patent Application Publication
(10) Pub. No.: US 2023/0186254 A1
(43) Pub. Date: Jun. 15, 2023

(54) OPTIMIZING METHOD FOR MULTI-SOURCE MUNICIPAL SOLID WASTE COMBINATIONS BASED ON MACHINE LEARNING

(71) Applicant: Tianjin University of Commerce, Tianjin (CN)

(72) Inventors: Guanyi CHEN, Tianjin (CN); Chengming DU, Tianjin (CN); Yunan SUN, Tianjin (CN); Junyu TAO, Tianjin (CN); Xinyi LIU, Tianjin (CN); Lan MU, Tianjin (CN); Xiaohua WANG, Tianjin (CN); Zhenyu WANG, Tianjin (CN); Yanni ZHENG, Tianjin (CN)

(73) Assignee: Tianjin University of Commerce, Tianjin (CN)

(21) Appl. No.: 17/984,514

(22) Filed: Nov. 10, 2022

(30) Foreign Application Priority Data
Dec. 10, 2021 (CN) 202111504631.3

(51) Int. Cl. G06Q 10/00; B09B 3/40; G06N 5/02

(52) U.S. Cl. CPC G06Q 10/30; B09B 3/40; G06N 5/022; B09B 2101/25

(57) ABSTRACT

Disclosed is an optimizing method for multi-source municipal solid waste combinations based on machine learning, including obtaining relevant property data, classifying the feature variables and obtaining a raw materials pre-combination from the classified feature variables according to a classification ratio, followed by cooperative combustion treatment to obtain data after combustion, summarizing the obtained data into a database, constructing a matrix of raw material components, operating conditions and pollutant distribution according to the database, obtaining matrix data; performing principal component analysis on the matrix data, constructing an information processing model, obtaining a data set of samples; carrying out training according to the data set to construct a relational model, obtaining processed parameters; training the obtained processed parameters to construct a regression module, an optimal parameter, and performing regression calculation using the optimal parameter together with the matrix data to obtain an optimization scheme of solid waste raw materials combinations.

### CROSS-REFERENCE TO RELATED APPLICATIONS

This application claims priority to Chinese Patent Application No. 202111504631.3, filed on Dec. 10, 2021, the contents of which are hereby incorporated by reference.

### TECHNICAL FIELD

The present application belongs to the technical field of multi-source municipal solid waste incineration treatment, and in particular to an optimizing method for multi-source municipal solid waste combinations based on machine learning.

### BACKGROUND

Solid wastes produced in the daily activities of urban residents are increasing as a result of the developed economy and the improved industrial production capacities in China, and concerns over how to safely and effectively dispose large amounts of multi-source urban solid waste (MSW) are surging. Among various methods for treating solid wastes, the method of incineration is preferred with advantages of quick volume reduction, effective oxidation and decomposition of most harmful substances in solid wastes, and recoverable heat energy; however, such a method no longer meets people's demand for environmental protection, and an incineration technology for solid wastes with higher requirements is expected in response to improved national environmental standards.

MSW is characterized by large amounts, multiple types, mixed components, scattered distribution and severe hazards, and feature pollutants including volatile organic compounds (VOCs) and heavy metals caused by solid waste disposal are more in need of effective control. As for treating MSW, a collaborative approach by using industrial kilns (rotary kiln, pulverized coal furnace, etc.) is proved, by existing research, to be effective in both reducing consumption of fossil fuel as well as cutting down emissions of greenhouse gases and other pollutants; yet, in the process of treating MSW, industrial kilns are required to be maintained with a stable temperature at inside, for molten slag, crusting and caking will be produced in the kilns if the temperature is too high, which have a great impact on the service life of industrial kilns; while combustion efficiency will be impaired if the temperature is too low, leading to an insufficient combustion of the solid waste and failure in effectively decomposing harmful substances; in fact, it is difficult to achieve a stable long-term operation of industrial kilns since feeding materials are varied greatly in nature in practice. Accordingly, appropriate combination schemes should be adopted at the source end according to the physical and chemical features of solid waste, so as to collaboratively treat solid waste and realize compatible matching of kiln processing system and thermotechnical process, in addition to effectively control of pollutant releasing. Therefore, it is necessary to develop an optimizing method for MSW combinations, which can not only ensure a stable operation of the industrial kilns in treating solid waste materials while reducing the generation of pollutants.

### SUMMARY

The present application aims to provide an optimizing method for multi-source urban solid waste (MSW) combinations based on machine learning; by applying machine learning algorithms in treating MSW combinations using industrial kilns, the method provides a guiding basis for actual combinations preparation, and ensures a stable operation of industrial kilns with effectively reduced pollutant emissions and improved economic benefits.

In order to achieve the above objectives, the present application provides an optimizing method for MSW combinations based on machine learning, including:

collecting samples of different kinds of solid wastes to obtain relevant property data;

screening and processing the relevant property data by a feature selection algorithm to obtain feature variables, classifying the feature variables according to modes of economy priority and emission priority, and obtaining a raw materials pre-combination from the classified feature variables according to a classification ratio;

subjecting the raw materials pre-combination to cooperative combustion treatment to obtain data after combustion, summarizing the obtained data into a database, then constructing a matrix of raw material components, operating conditions and pollutant distribution according to the database, and obtaining matrix data;

performing principal component analysis on the matrix data, constructing an information processing model, and obtaining a data set of samples;

carrying out training according to the data set to construct a relational model, and obtaining processed parameters; and

training the obtained processed parameters to construct a regression module, an optimal parameter, and performing regression calculation using the optimal parameter together with the matrix data to obtain an optimization scheme of solid waste raw materials combinations.

Optionally, the relevant property data include properties of elemental compositions, thermal weight loss features, component features and heat values of the samples.

Optionally, the relevant property data are obtained through a thermogravimetric (TG) analyzer and an infrared spectrometer.

Optionally, the feature variables are classified with a pre-requisite of constructing a classification module model, where the classification module model is constructed as follows: performing vector classification of the feature variables screened out according to modes of economy priority and emission priority, obtaining classification parameters, carrying out optimization, and constructing the classification module model.

Optionally, the ratio for raw materials pre-combination is obtained according to types of raw materials, and existing national industrial standards.

Optionally, the information processing module is used to perform dimension reduction and noise reduction of the data, so as to obtain several principal components, where the principal components contain original data information and are not related to each other, and a first five percent of the principal components are extracted for subsequent analysis and calculation.

Optionally, the optimization of MSW combinations includes a process as follows: training the model with data of a training group according to emission data of SOx, NOx and other pollutants and a number of different principal components, and then predicting the pollutants of the samples in the combination in terms of emission with a data of a analysis test group in the model, and evaluating predicted results with an average relative error to obtain an optimized model.

Optionally, the regression calculation includes a process as follows: obtaining matrix data of raw material components, operating conditions and pollutant distribution acquired based on a collecting module, processing the obtained data using the information processing module, and inputting the data into a regression module model for regression calculation to obtain the heat value of the samples combinations and results of pollutants emission.

The present application achieves technical effects below:

firstly, providing an optimizing method for multi-source MSW combinations that enables effectively improved energy recovery of MSW as well as different combination schemes that meet the requirements of various strategies in the actual solid waste treatment;

secondly, enabling substantial reduction of pollutants emission in the traditional process of solid waste treatment; and

thirdly, effectively improving the resource utilization rate of MSW in the application of co-treatment of MSW together with industrial kilns.

### BRIEF DESCRIPTION OF THE DRAWINGS

The drawings that form a part of this application are used to provide a further understanding of this application. The illustrative embodiments of this application and their descriptions are used to explain this application, and do not constitute undue limitations on this application. In the attached drawings:

FIG. 1 shows a process of an optimizing method for MSW combinations provided in one Embodiment of the present application.

FIG. 2 is a schematic diagram illustrating a structural composition of the optimizing method for MSW combinations provided in one Embodiment of the present application.

FIG. 3 is a processing illustrating solid waste samples combinations in regard of economic priority.

FIG. 4 is a processing illustrating solid waste samples combinations in regard of emission priority.

### DETAILED DESCRIPTION OF THE EMBODIMENTS

It should be noted that the embodiments in this application and the features in the embodiments may be combined with each other without conflict. The application is described in detail with reference to the drawings and embodiments.

It should also be noted that the steps shown in the process of the drawings can be executed in a computer system such as a set of computer-executable instructions, and, although a logical sequence is shown in the process, in some cases, the steps shown or described may be executed in a sequence different from that here.

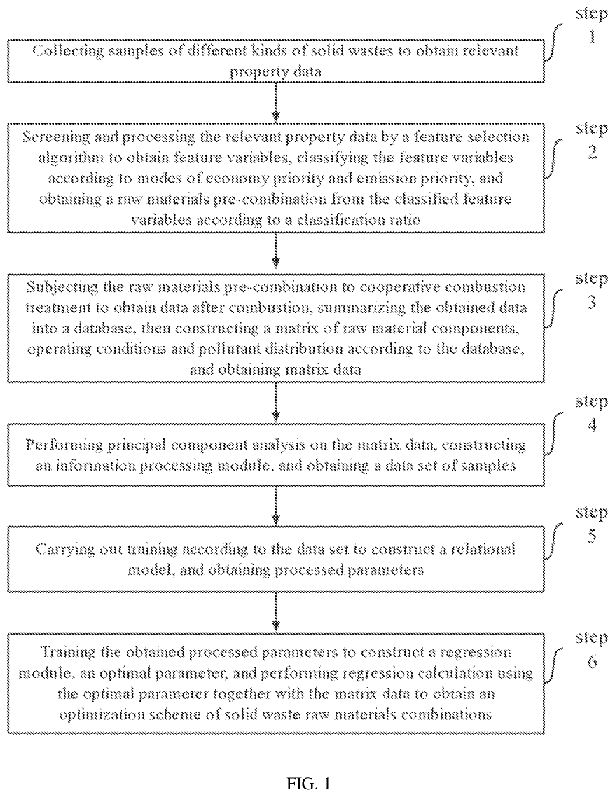

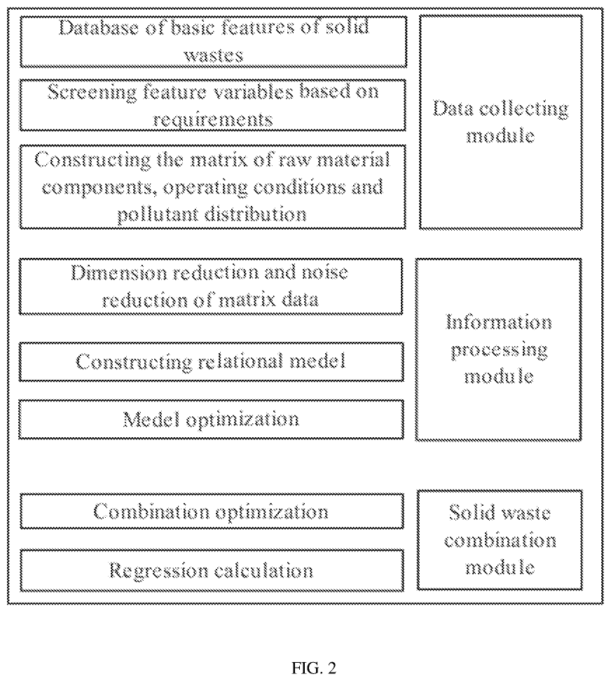

As shown in FIGS. 1-2, this embodiment provides an optimizing method for MSW combinations based on machine learning, including:

step 1, collecting samples of different kinds of solid wastes to obtain relevant property data;

step 2, screening and processing the relevant property data by a feature selection algorithm to obtain feature variables, classifying the feature variables according to modes of economy priority and emission priority, and obtaining a raw materials pre-combination from the classified feature variables according to a classification ratio;

step 3, subjecting the raw materials pre-combination to cooperative combustion treatment to obtain data after combustion, summarizing the obtained data into a database, then constructing a matrix of raw material components, operating conditions and pollutant distribution according to the database, and obtaining matrix data;

step 4, performing principal component analysis on the matrix data, constructing an information processing module, and obtaining a data set of samples;

step 5, carrying out training according to the data set to construct a relational model, and obtaining processed parameters; and

step 6, training the obtained processed parameters to construct a regression module, an optimal parameter, and performing regression calculation using the optimal parameter together with the matrix data to obtain an optimization scheme of solid waste raw materials combinations.

In another embodiment, the relevant property data include properties of elemental compositions, thermal weight loss features, component features and heat values of the samples.

In another embodiment, the relevant property data are obtained through thermogravimetric (TG) analyzer and infrared spectrometer.

In another embodiment, the feature variables are classified with a pre-requisite of constructing a classification module model, where the classification module model is constructed as follows: performing vector classification of the feature variables screened out according to modes of economy priority and emission priority, obtaining classification parameters, carrying out optimization, and constructing the classification module model.

In another embodiment, the ratio for raw materials pre-combination is obtained according to types of raw materials, and existing national industrial standards.

In another embodiment, the information processing module is used to perform dimension reduction and noise reduction of the data, so as to obtain several principal components, where the principal components contain original data information and are not related to each other, and a first five percent of the principal components are extracted for subsequent analysis and calculation.

In another embodiment, the optimization of MSW combinations includes a process as follows: training the model with data of a training group according to emission data of SOx, NOx and other pollutants and a number of different principal components, and then predicting the pollutants of the samples in the combination in terms of emission with a data of a analysis test group in the model, and evaluating predicted results with an average relative error to obtain an optimized model.

In another embodiment, the regression calculation includes a process as follows: obtaining matrix data of raw material components, operating conditions and pollutant distribution acquired based on a collecting module, processing the obtained data using the information processing module, inputting the data into a regression module model for regression calculation to obtain the heat value of the combination sample and result of pollutant emission.

Specifically, step 1 includes: collecting a large number of samples of different kinds of solid wastes from various areas and storing them under specific conditions; meanwhile, establishing a database of basic features of solid wastes according to experimental data, which mainly includes elemental composition, thermal weight loss features, component features, heat values and other features of solid wastes;

step 2 specifically includes: treating the solid waste samples in different degrees according to using requirements of a machine, collecting TG-Fourier Transform Infrared (FTIR) data of the samples by a data collecting module composed of a TG analyzer and an infrared spectrometer;

step 3 specifically includes: using Boruta feature selection algorithm in R language to screen out the feature variables with priorities of economy and different emissions of solid waste combinations from the database of basic features of solid wastes; training the classification module model based on supporting vector classification, neural network, etc. according to the screened important features data, optimizing an objective function with accuracy rate, recall rate or other parameters and indicators, obtaining the best parameters of the module model to deliver an optimal classification module model of solid waste; mixing a same kind of solid wastes in different proportions according to results of classification for combinations, carrying out co-combustion experiment according to the combination prediction, summarizing the experimental data to form a database, then constructing the matrix of raw material components, operating conditions and pollutant distribution;

step 4 specifically includes: using Scikit-learn v0.21.2 package in Python 3.7.3 programming environment and adopting principal component analysis algorithm to construct an information extraction module model; inputting matrix data of raw material composition, operating conditions and pollutant distribution in step 3 into the information extraction module model, and the data are subjected to dimension reduction and noise reduction to obtain several principal components, where the principal components contain original data information and are not related to each other, and the first five percent of the extracted data is used for subsequent analysis and calculation;

step 5 specifically includes: training the regression module model based on support vector regression, random forest and the like by using the processed sample data obtained in step 4, and taking the average relative error or other parameter indexes as optimization objective functions to obtain the best parameter conditions of the module model and generate the best regression module models, where each regression module corresponds to only one test item, including the heat value of raw materials combinations, the emission of pollutant NOx, the emission of pollutant SOx, etc., and several different regression models should be trained for analyzing different items; training the processed sample data obtained in step 4 for support vector regression, and using the average relative error as the optimization objective function to obtain the optimal parameter conditions of the module model, and developing the optimal regression module model diagram for predicting low heat value; constructing model using the support vector regression algorithm, and adopting three kernel functions of the support vector machine, including Linear kernel function, radial basis kernel function (RBF) and polynomial kernel function Poly; dividing 4 samples of each mixing material yet different mixing ratios into training group and testing group; training the model using data of training group according to the emission data of SOx, NOx and other pollutants and the number of different principal components, then using the model to analyse data of the testing group to predict the pollutant emission of the samples combinations, and evaluating the predict results using average relative error obtain the optimized model; and

step 6 specifically includes: obtaining the matrix data of raw material components, operating conditions and pollutant distribution for new solid waste raw material combination by the data collecting module in step 3, and obtaining processed data through information extraction module in step 4, inputting the data into the regression module model obtained in step 5 for regression calculation, obtaining results of heat values and pollutant emissions of the samples combinations, and optimizing scheme of solid waste raw material combination according to the results.

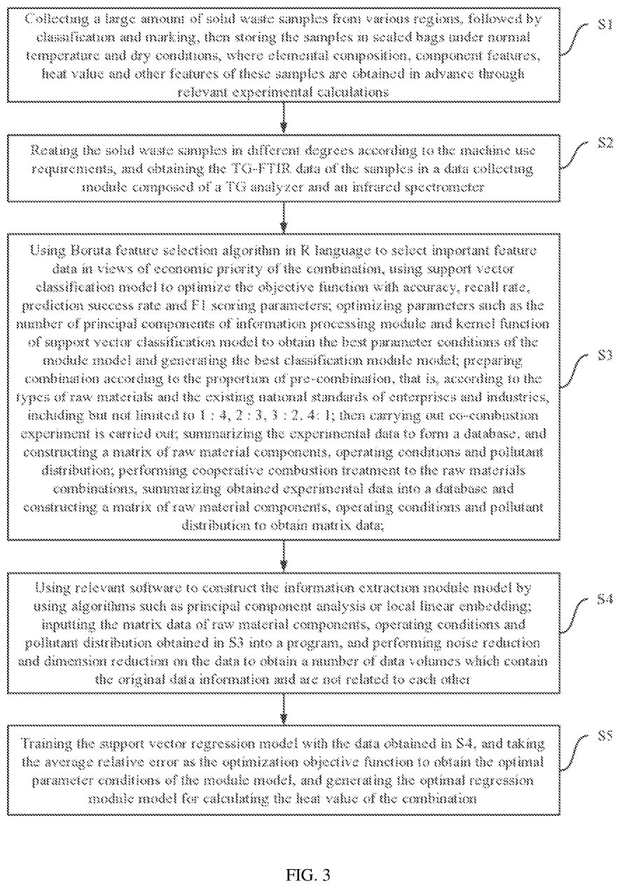

See FIG. 3 for solid waste samples combinations in regard of economic priority, including:

S1, collecting a large amount of solid waste samples from various regions, followed by classification and marking, then storing the samples in sealed bags under normal temperature and dry conditions, where elemental composition, component features, heat value and other features of these samples are obtained in advance through relevant experimental calculations;

S2, treating the solid waste samples in different degrees according to the machine use requirements, and obtaining the TG-FTIR data of the samples in a data collecting module composed of a TG analyzer and an infrared spectrometer;

In this embodiment, the TG infrared experiment adopts a temperature rising rate of 20 degree Celsius per minute (° C./min), with initial temperature of room temperature-1,000° C., and air is used as a combustion gas to simulate the combustion process, with air flow rate being set at 80 milliliters per min (mL/min); a connecting pipe between the TG analyzer and the infrared spectrometer and a gas pool are both pre-heated to 180° C. before the experiment started; the infrared spectrometer has a scanning wave range of 400-4,000 reciprocal centimeter (cm⁻¹), with resolution being set to 0.482 cm⁻¹;

S3, using Boruta feature selection algorithm in R language to select important feature data in views of economic priority of the combination, where the economic priority specifies that the heat value of solid waste combination should meet the requirements of kiln design as much as possible to reduce the amount of auxiliary fuel, and the heat value should be controlled at about 3,000-5,000 kilocalorie per kilogram (kcal/kg) to ensure the economic and reliable operation of the system; in feature training, using support vector classification model to optimize the objective function with accuracy, recall rate, prediction success rate and F1 scoring parameters; optimizing parameters such as the number of principal components of information processing module and kernel function of support vector classification model to obtain the best parameter conditions of the module model and generating the best classification module model; preparing combination according to the proportion of pre-combination, that is, according to the types of raw materials and the existing national standards of enterprises and industries, including but not limited to 1:4, 2:3, 3:2, 4:1; then carrying out co-combustion experiment is carried out; summarizing the experimental data to form a database, and constructing a matrix of raw material components, operating conditions and pollutant distribution;

performing cooperative combustion treatment to the raw materials combinations, including TG-FTIR test and small-scale constant-temperature settling furnace test, so as to obtain comprehensive combustion feature index, sulfur oxide emission concentration, carbon monoxide emission concentration, nitrogen oxide emission concentration, dioxin emission concentration and heavy metal emission concentration under different working conditions, summarizing the obtained experimental data into a database and constructing a matrix of raw material components, operating conditions and pollutant distribution to obtain matrix data;

S4, using relevant software to construct the information extraction module model by using algorithms such as principal component analysis or local linear embedding; inputting the matrix data of raw material components, operating conditions and pollutant distribution obtained in S3 into a program, and performing noise reduction and dimension reduction on the data to obtain a number of data volumes which contain the original data information and are not related to each other;

in this embodiment, the Scikit-learn v0.21.2 package is used in Python 3.7.3 programming environment, and the principal component analysis algorithm is adopted to construct the information extraction module model; and the data obtained in S2 is input into this model and subjected to dimension reduction and noise reduction, with the top five percent of the data being extracted for subsequent analysis and calculation; and

S5, training the support vector regression model with the data obtained in S4, and taking the average relative error as the optimization objective function to obtain the optimal parameter conditions of the module model, and generating the optimal regression module model for calculating the heat value of the combination.

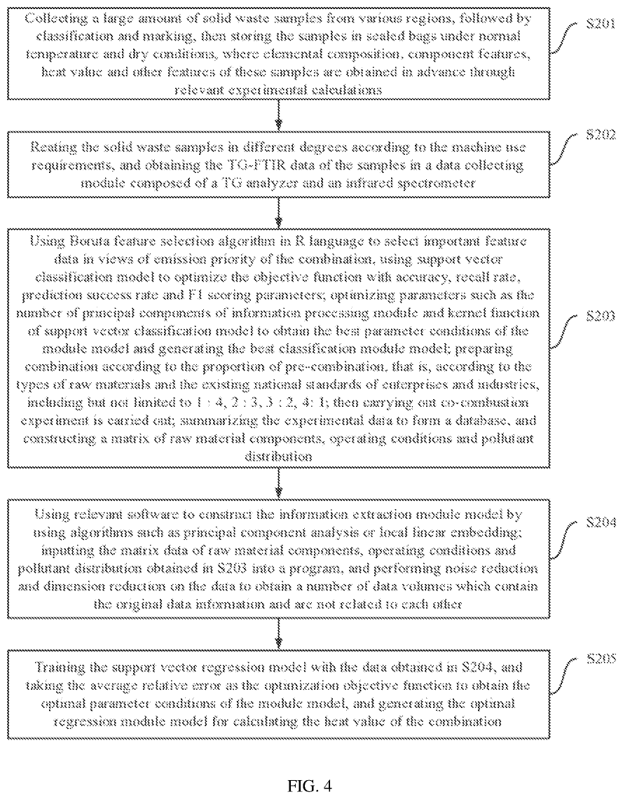

See FIG. 4 for solid waste samples combinations in regard of emission priority, including:

S201, collecting a large amount of solid waste samples from various regions, followed by classification and marking, then storing the samples in sealed bags under normal temperature and dry conditions, where elemental composition, component features, heat value and other features of these samples are obtained in advance through relevant experimental calculations;

S202, treating the solid waste samples in different degrees according to the machine use requirements, and obtaining the TG-FTIR data of the samples in a data collecting module composed of a TG analyzer and an infrared spectrometer;

in this embodiment, the TG infrared experiment adopts a temperature rising rate of 20° C./min, with initial temperature of room temperature-1,000° C., and air is used as a combustion gas to simulate the combustion process, with air flow rate being set at 80 mL/min; a connecting pipe between the TG analyzer and the infrared spectrometer and a gas pool are both pre-heated to 180° C. before the experiment started; the infrared spectrometer has a scanning wave range of 400-4,000 cm⁻¹, with resolution being set to 0.482 cm⁻¹;

S203, using Boruta feature selection algorithm in R language to select important feature data in views of emission priority of the combinations, where the emission priority focuses on the emission concentration of typical pollutants such as SOx and NOx, the emission concentration of volatile elements and substances (Pb, Cd, As, alkali metal compounds, alkali metal sulfates, etc.) and the emission concentration of heavy metals (Cr, Ni, Mn, etc.); in feature training, using support vector classification model to optimize the objective function with accuracy, recall rate, prediction success rate and F1 scoring parameters; optimizing parameters such as the number of principal components of information processing module and kernel function of support vector classification model to obtain the best parameter conditions of the module model and generating the best classification module model; preparing combination according to the proportion of pre-combination, that is, according to the types of raw materials and the existing national standards of enterprises and industries, including but not limited to 1:4, 2:3, 3:2, 4:1; then carrying out co-combustion experiment is carried out; summarizing the experimental data to form a database, and constructing a matrix of raw material components, operating conditions and pollutant distribution;

S204, using relevant software to construct the information extraction module model by using algorithms such as principal component analysis or local linear embedding; inputting the matrix data of raw material components, operating conditions and pollutant distribution obtained in S203 into the program, and performing noise reduction and dimension reduction on the data to obtain a number of data volumes which contain the original data information and are not related to each other; and

S205, training the regression module model based on support vector regression, random forest, etc. by using the data obtained in S204, taking the average relative error or other parameter indexes as the optimization objective function to obtain the optimal parameter conditions of the module model and generating the optimal regression module model, where each regression module corresponds to only one test item, including the emission concentrations of NOx and SOx, emission concentrations of heavy metal Pb etc., and several different regression models should be trained for analyzing different items.

The test item in this embodiment is emission concentration of NOx, and the model is constructed by using support vector regression algorithm, with three kernel functions of support vector machine adopted, including: Linear kernel function, RBF and polynomial kernel function Poly; 4 samples of each mixing material yet with different mixing ratios are divided into training group and testing group; the model is trained using data of training group according to the emission data of NOx and the number of different principal components, then the model is used to analyse data of the testing group to predict the pollutant emission of the samples combinations, and the predict results are evaluated using average relative error obtain the optimized model.

The present application provides an optimizing method for multi-source MSW combinations, which can be used to effectively improve the energy recovery of MSW, and realize the output of combination schemes that meet various strategic requirements in actual solid waste treatment accordingly; the method can also be applied in traditional solid waste treatment and therefore enable effectively reduction of pollutants emission during solid waste treatment; and together with industrial kilns, the method provides an effectively improved utilization rate of MSW in the field of co-treatment of MSW.

The above are only the preferred embodiments of this application, but the scope of protection of this application is not limited to this. Any changes or substitutions that can be easily thought of by those skilled in the technical field within the technical scope disclosed in this application should be covered by the scope of protection of this application. Therefore, the scope of protection of this application should be based on the scope of protection of the claims.

What is claimed is:

1. An optimizing method for multi-source municipal solid waste (MSW) combinations based on machine learning, comprising:
collecting samples of different kinds of solid wastes to obtain relevant property data;
screening and processing the relevant property data by a feature selection algorithm to obtain feature variables, classifying the feature variables according to modes of economy priority and emission priority, and obtaining a raw materials pre-combination from the classified feature variables according to a classification ratio;
subjecting the raw materials pre-combination to cooperative combustion treatment to obtain data after combustion, summarizing the obtained data into a database, then constructing a matrix of raw material components, operating conditions and pollutant distribution according to the database, and obtaining matrix data;
performing principal component analysis on the matrix data, constructing an information processing module, and obtaining a data set of samples;
carrying out training according to the data set to construct a relational model, and obtaining processed parameters; and
training the obtained processed parameters to construct a regression module, an optimal parameter, and performing regression calculation using the optimal parameter together with the matrix data to obtain an optimization scheme of solid waste raw materials combinations.

2. The optimizing method for multi-source municipal solid waste combinations based on machine learning according to claim 1, wherein the relevant property data comprises properties of elemental compositions, thermal weight loss features, component features and heat values of the samples.

3. The optimizing method for multi-source municipal solid waste combinations based on machine learning according to claim 2, wherein the relevant property data are obtained through a thermogravimetric (TG) analyzer and infrared spectrometer.

4. The optimizing method for multi-source municipal solid waste combinations based on machine learning according to claim 1, wherein the feature variables are classified with a prerequisite of constructing a classification module model, where the classification module model is constructed according to following steps: performing vector classification of the feature variables screened out according to modes of economy priority and emission priority, obtaining classification parameters, carrying out optimization, and obtaining the classification module model.

5. The optimizing method for multi-source municipal solid waste combinations based on machine learning according to claim 4, wherein the classification ratio for raw materials pre-combination is obtained according to types of raw materials, and existing national industrial standards.

6. The optimizing method for multi-source municipal solid waste combinations based on machine learning according to claim 1, wherein the information processing module is used to perform dimension reduction and noise reduction of the data, so as to obtain several principal components, where the principal components contain original data information and are not related to each other, and a first five percent of the principal components is extracted for subsequent analysis and calculation.

7. The optimizing method for multi-source municipal solid waste combinations based on machine learning according to claim 1, wherein the optimization of MSW combinations comprises a process as follows: training the model with data of a training group according to emission data of SOx, NOx and other pollutants and a number of different principal components, and then predicting the pollutants of the samples in the combination in terms of emission with a data of a analysis test group in the model, and evaluating predicted results with an average relative error to obtain an optimized model.

8. The optimizing method for multi-source municipal solid waste combinations based on machine learning according to claim 1, wherein the regression calculation comprises a process as follows: obtaining matrix data of raw material components, operating conditions and pollutant distribution acquired based on a collecting module, processing the obtained data using the information processing module, inputting the data into a regression module model for regression calculation to obtain the heat value of the combination sample and result of pollutant emission.
